(19) United States
(12) Patent Application Publication
(10) Pub. No.: US 2022/0233435 A1
(43) Pub. Date: Jul. 28, 2022

(54) CONTROLLED RELEASE DEVICE FOR ORAL CAVITY

(71) Applicant: Capsamedix Oy, Helsinki (FI)

(72) Inventors: Antti Rahikkala, Helsinki (FI); Mohammad-Ali Shahbazi, Helsinki (FI); Hélder A. Santos, Helsinki (FI)

(21) Appl. No.: 17/610,258

(22) PCT Filed: May 14, 2020
(86) PCT No.: PCT/FI2020/050322

§ 371 Date: Nov. 10, 2021

(30) Foreign Application Priority Data
May 14, 2019 (FI) 20195397

(51) Int. Cl. A61K 9/00; A61K 47/32; A61K 47/34

(52) U.S. Cl. CPC A61K 9/0053; A61K 47/34; A61K 47/32

(57) ABSTRACT

The present invention relates to a controlled release device for oral cavity, which is attached on a hard dental surface of a tooth. The attachment is done using any adhesive layer that can attach on enamel surface of a tooth or using a holder attached to the hard dental surface, to which holder the device can be attached, clipped or fastened. The device comprises of two or more polymeric materials forming a polymer matrix or matrices. The polymer matrices incorporate one or more agent(s), which release rate in the oral cavity can be con-trolled with the polymeric materials in the matrices. The invention also relates to a method producing a controlled re-lease device for delivering an agent to the oral cavity.

### FIELD OF THE INVENTION

The present invention relates to a controlled release device for releasing an active agent. The active ingredient can be a drug, an active pharmaceutical or supplemental ingredient, etc., which is released in a controlled sustained manner to achieve a local or systemic physiological or pharmacological effect. More particularly, the invention relates to a controlled release device comprising a polymer matrix containing the active agent. The controlled release device is attached on the dental enamel (hard dental) surface and releases the active agent directly into the oral cavity in a controlled sustained manner.

### BACKGROUND OF THE INVENTION

Currently, there is a lack of devices and methods that allow controlled local release of active agents in the oral cavity, especially in a long-term, sustained fashion. Traditional options for oral drug release are, for example, a pill or lozenge dissolving in the mouth and the subsequent absorption of active pharmaceutical ingredient (API) molecules into the mucosal tissue, from where they enter into the blood circulation, or are swallowed in order to enter the gastrointestinal tract. In most of the cases, a non-sustained API release occurs, and the therapeutic effect is reduced in minutes or hours.

Previously reported related applications for oral delivery include teeth-planted sensors and oral drug delivery devices, such as polymeric tooth-guards and films or patches that can be placed on the mucosa. Tooth-guards have a high volume and, as such, are capable of delivering large amounts of APIs; however, wearing such a device is very burdensome for users, especially for long time duration. Thin patches cannot accommodate large amounts of APIs, and especially the attachment on mucosa is challenging and questionable, as the soft tissue may rupture under mechanical stress, which leads to the detachment of drug delivery device and failed treatment. Furthermore, the mechanisms behind mucoadhesion are not well-known, and it may include penetration of polymers inside mucus and drying of the mucosal tissue in the contact area.

Publication U.S. Pat. No. 6,197,331 describes a controlled-release solid composition for the oral cavity that adheres to hard dental surface, in which the composition includes an adhesive component, a polymer, a plasticizer and one or more pharmaceutically active agents.

Patent publication WO2011001425 describes an oral delivery device for the treatment of periodontal disease. The device is a solid unit dosage form configured for insertion into a periodontal pocket of a patient. The device consists of a biodegradable pharmaceutically acceptable water-insoluble polymer and a therapeutic effective amount of an anti-inflammatory agent. The device can further include an enzymatically biodegradable pharmaceutically acceptable water soluble polymer.

### BRIEF DESCRIPTION OF THE INVENTION

An object of the present invention is to provide a controlled release device for improved release of active agents or active pharmaceutical ingredients in the oral cavity.

The invention therefore provides a controlled release device for delivering an agent to the oral cavity, wherein the device comprises
one or more polymer matrices,
each polymer matrix comprises one or more polymeric materials,
at least one polymer matrix comprises an agent that can be released from said matrix in a controlled manner,
means for attaching said device to a hard dental surface.

The invention further provides a method of producing a device according to the invention, wherein the method comprises the steps of
forming the polymer matrix from at least one polymeric material,
adding to said polymer matrix an agent,
optionally adding further components to the polymer matrix and
optionally forming the polymer matrix including the agent to a capsule, a disc, a tablet, a cartridge, a pellet, a veneer or a troche.

The invention still further provides a method of producing a device according to the invention, wherein the method comprises the steps of
incorporating the agent into the polymeric material during formation of the polymer matrix from at least one polymeric material,
optionally adding further components to the polymer matrix and
optionally forming the polymer matrix including the agent to a capsule, a disc, a tablet, a cartridge, a pellet, a veneer or a troche.

Currently, there is a lack of devices and methods that allow sustained drug release for localized medication in oral cavity. Thus, developing a device or method capable of maintaining the sustained release of agents from days to several weeks or months will have a huge impact on: (i) the treatment of certain medical conditions that require local medication in mouth and/or sustained release over time; (ii) the patient welfare due to decreased amount of applied therapy/medication; and (iii) also in applications where maintaining the end-user's basic health is important, such as in oral hygiene and nicotine cessation.

As such, the most durable and harmless spots for orally planted drug delivery devices are the teeth. A solid polymeric tooth-planted device offers increased API capacity compared to thin patches, provides a user-friendly experience as it can be designed small enough to be non-sensible. In addition, different tooth attachment strategies can be employed, ranging from dental adhesives to device-integrated adhesive layer, holder mediated attachment, clipping or fastening of the device on the hard dental surface of the tooth for fast and easy personalized removal and attachment, as well as magnetic holding of the device on the tooth. In all these cases, a polymeric composition of the device can be tuned for different compound release rates and time duration.

Thus, developing a device or method capable of maintaining sustained drug release for days or several weeks will have a huge impact on the treatment of certain medical conditions that require local medication in mouth, on patient welfare due to decreased amount of applied therapy/medication, and also in applications where maintaining of end-user's basic health is important, such as in oral hygiene and nicotine cessation.

The device according to the invention allows long steady-state agent release for many potential applications, while simultaneously being non-sensible for the user. Lozenges and similar have a high initial burst release, resulting in a need to repeatedly apply the medication (e.g. on an hourly basis). This can be problematic, for example, due to strict limitations on the daily dose. Furthermore, for medical conditions localized in the oral cavity, currently the common treatments are the said lozenges and mouth rinses, which may be painful and irritating due to the need of repeated doses. They also do not remain for long time in the oral cavity, thus further increasing the demand for repeated doses. Mucoadhesive patches may have questionable bonding strength to the tissue and probably cannot accommodate high enough amounts of active agents. Tooth-guards are extremely uncomfortable to wear and cannot be used for long-term medication. In practice, toothguard can also be worn during sleeping and would compromise the dosing of the active agent during awake time.

### BRIEF DESCRIPTION OF THE DRAWINGS

In the following, the invention will be described in greater detail by means of preferred embodiments with reference to the attached [accompanying] drawings, in which

FIG. 1 illustrates an example of the cumulative release of acetaminophen (paracetamol) from a controlled release device vs time (days). The maximum calculated drug payload is 1 mg. The labels indicate the weight percentage of poly(vinyl alcohol) (PVA) in the polymer matrix formulation, for example, PVA 25% consists of 25 w-% PVA and 75 w-% poly(lactide) (PLA).

FIG. 2 illustrates an example of the cumulative release of hydroxycitric acid (HCA) originating from Garcinia Cambogia herbal extract loaded in a controlled release device vs time (days). The results are given as the amount of Garcinia Cambogia extract that the HCA originates from. The labels indicate the weight percentage of poly(vinyl alcohol) (PVA) in the polymer matrix formulation, for example, PVA 10% consists of 10 w-% PVA and 90 w-% poly(lactide) (PLA).

FIG. 3 illustrates an example of the cumulative releases of nicotine and vanillin from a controlled release device vs time (days). The maximum calculated payload of nicotine and vanillin is 5.75 mg and 2.1 mg, respectively. The polymer matrix formulation contain 50 w-% of PVA and 50 wt-% PLA.

FIG. 4 illustrates an example of the controlled release device, as follows

FIG. 4A illustrates the controlled release device with a view of the bottom surface, where a and b denote the dimensions of the device;

FIG. 4B illustrates the controlled release device with a view of the longer side surface, where b and c denote dimensions of the device;

FIG. 4C illustrates the controlled release device with a view, where the device is tilted such that dimensions a, b and c are visible.

### DETAILED DESCRIPTION OF THE INVENTION

The invention provides a controlled release device for releasing an agent into the oral cavity. The controlled release device comprises one or more polymer matrix (matrices). The one or more polymer matrices comprise one or more polymeric material(s). At least one polymer matrix comprises an agent that can be released from the matrix in a controlled manner into the oral cavity. The polymer matrix can also comprise more than one agent. More than one polymer matrix can comprise an agent, which can be the same or different. Multiple agents can also be released simultaneously from one device.

The controlled release device in addition comprise means to attach the device to the hard dental surface, i.e. enamel of a tooth. Several devices can be used at the same time on the same tooth or different teeth, allowing better control of dosage amount needed for the patients (depending on age, sex, disease state, etc).

The controlled release device comprises one or more polymer matrices. Each polymer matrix can be formed as a layer, patch or film. Especially when the device is formed as a patch or disc, the device can be formed from separate layers. The separate layers can be composed of different polymer matrices or the layers can be formed from the same polymer matrix formed in separate layers or films. Two polymer matrices can also be mixed or formed in any manner when forming the controlled release device. If a multi-layered device is fabricated, the layers are formed from different polymer matrices or the same polymer matrices. The device can contain varied number of layers, for example from 2 to 10 layers, preferably from 3 to 6 layers.

The polymer matrix constituting the controlled release device comprises at least one polymeric material. The polymer matrix can also comprise other compounds, such as plasticizing agents or other additives to the polymeric material. The polymer matrix can also comprise other materials that promote the properties of the device, such as softeners, hygroscopic compounds, mucosal penetration or permeation enhancers, and surface enhancing components or surface coatings. The polymer matrices can comprise a copolymer, such as poly(ethylene-vinyl acetate for adjusting the mechanical properties and softness of the device.

According to one embodiment, the polymer matrices comprise at least two polymeric materials of which a first polymer material is a water insoluble polymer and a second polymer material is a water soluble polymer. The term “soluble” is here given its normal meaning. Water solubility is a well-established property of various chemical components and compositions. Water insoluble polymer here means a polymer with a solubility of <1 mg/mL water. Water soluble polymer here means a polymer with a solubility of ≥1 mg/mL water.

The water insoluble polymer can be any polymer not soluble in water, but preferably all material used should be approved for medical use and be biodegradable. The water insoluble polymer can be either swellable or non-swellable within the oral cavity, such as urethane- and acrylate-based polymers. Examples of suitable water insoluble polymers are poly(caprolactone) (PCL) and poly(lactide) (PLA) but can be other polymers. Examples of suitable water soluble polymers are poly(vinylpyrrolidone) (PVP) and poly(vinyl alcohol) (PVA).

The agent, which is comprised in the at least one polymer matrix and can be any active molecule, is released due to diffusion to oral cavity, swelling of the device, decomposition or dissolution of the polymer matrix. For example, if a 100% PLA polymer matrix formulation would be used, the release of the agent would happen by decomposition due to hydrolysis of the polymer chain. On the other hand, if a 100% PVA polymer matrix formulation would be used, dissolution of the polymer will cause faster drug release from the polymer matrix. When a combination of at least two polymer materials with different water solubility properties is used, the release of the agent can be controlled by varying the weight ratio of the two polymeric materials. The materials need to be biocompatible and biodegradable, because the principle of release inevitably leads to small amounts of polymer transition to the gastrointestinal (GI) tract by swallowing of the polymer components over time.

According to one embodiment of the invention, the weight ratio of the first polymeric material to the second polymer material is from 9:1 to 1:9, preferably from 8:2 to 2:8 and most preferably from 6:4 to 4:6. This is dependent on the desired release rate of active agent, which is in close relation to the purpose of device development, type of disease, and the duration of the therapy.

The term “weight percentage” (w-%) as used herein and hereafter in context of weight percentage of polymeric material(s) in a polymer matrix formulation denotes the weight percentages of only the polymeric material(s) in said polymer matrix formulation. Therefore, the weight(s) of the one or more agent(s) is/are not considered when the weight percentage of polymeric material(s) in a polymer matrix formulation are used. For example, PVA 25 w-% denotes that the polymer matrix formulation comprises 25 w-% PVA, 75 w-% of a second polymeric material (e.g. PLA), and in addition to the polymer materials the polymer matrix can then contain one or more agents. It is to be understood that a polymer matrix formulation denotes a polymer matrix comprising one or more polymeric material(s) and in addition comprises one or more agent(s).

According to one embodiment of the invention, the polymer matrix also comprises one or more further components. The further components can be plasticizing agents or other additives to the polymeric material. The polymer matrix can also comprise other materials that promote the properties of the device, such as softeners and surface enhancing components or surface coatings. The polymer matrices can comprise a copolymer, such as poly(ethylene-vinyl acetate) for softening the material. Alternatively, the polymer matrix can comprise nanoparticles and preferably the agent or any other component can be encapsulated into the incorporated nanoparticle.

The controlled release device also comprises means for attaching the device to the hard dental surface. With the term “hard dental surface” is meant any part of the tooth on which a device according to the invention can be attached (both personalized device and surface flat device). The device can be fitted outside (anterior) area of the tooth, inside (posterior) area of the tooth or the top (coronal) area of the tooth as a dental crown or veneer. In practice, the attachment is performed on the enamel of the tooth.

The device can be attached to one side of a tooth, or various sides of the tooth or it can be attached to several teeth. Preferably, the device is attached to the out side (anterior) of one or more tooth. If controlled release devices are attached to more than one tooth, the devices do not need to be the same and/or can contain different agents. For example, a device with a fast release of one agent and a device with a slower release of another or same agent can be attached to the teeth of the same individual at the same time.

The controlled release devices according to the invention can be used on any teeth. The device can be used on humans and animals. The device can be used on pet animals, such as cats, dogs, and rodents, and it can also preferably be used on farm animals such as cows, pigs and horses.

The means for attaching the controlled release device to a hard dental surface can be any suitable way of attaching a polymeric material or matrix to the dental surface.

The means for attaching the device to the dental surface can be performed by using an adhesive material as one of the polymer matrices of the device. The polymer matrix that function as an adhesive material then forms a layer on the controlled release device and can attach the device to the hard dental surface. The adhesive can be any adhesive material capable of forming an adhesive layer between the enamel of the tooth and the polymer material of the device. The adhesive material can be an FDA and/or EMA approved biocompatible adhesive material. The adhesive layer, attaching the device to the dental surface, can be one of the polymer matrices of the device for controlled release of an active agent. If a polymer matrix of the controlled release device forms the adhesive layer, the polymer matrix can be a biopolymer film or hydrogel capable of attaching the device to the dental surface.

The means for attaching the device to the dental surface can also be formed as a separate dental adhesive to be used together with the device. Any suitable dental adhesive can be used. Dental adhesives are well-known for the person skilled in the art. For example, the dental adhesive can be a polymer resin, a composite material or UV curable dental resin adhesives.

The dental resin adhesive is applied on the enamel surface of the tooth, whereupon the device is fastened. The resin to attach the device may be self-etching or may include separate etching, priming, and bonding steps, and as such, the bonding kit can include separate reagents. For an application where users can attach the device themselves, a self-etching single-reagent adhesive is preferred. The adhesive can be composed of, for example, hydroxylethyl methacrylate (HEMA) and/or bisphenol glycidyl methacrylate. The polymerization of the adhesive may be self-cured or light cured, often with UV-light.

Alternatively, the means for attaching the device to the dental surface can be using a holder, which is attached to the hard dental surface and to which holder the controlled release device can be attached, clipped or fastened. The holder is attached to the tooth using any dental adhesive resin or composite. The controlled release device or polymer matrix is then attached to the holder. The holder itself can also be flexible and can have protruding parts on which the controlled release device can be clipped onto and thereby attached to the holder. The holder itself can be biodegradable but should not dissolve or decompose as fast as the device attached to the holder. The holder can therefore be used for several controlled release devices, without the need to attach each device separately to the dental surface.

For example, the holder can be designed to last for days, weeks or up to months at a time, and the controlled release device can be designed to last from minutes and hours to a day or several days or months, depending on the purpose of the design. For example, a design where the same holder can be used for a month and a new controlled release device is applied once or twice per day can be used. The duration of the holder versus the duration of the device depends on the agent used in the device and the person skilled in the art can design a suitable administration pattern for the devices and the agents.

The device can be attached or fastened to the holder by any suitable means. For example, the holder can be formed having flexible clips or claws on which the device can be fastened by pressing against the holder. The holder can also contain grooves or slits on where the device can be inserted or on which the device can slide on. The holder can also have magnetic properties to hold the device on the tooth without detachment for a certain period of time.

The adhesive layer should be strong and durable enough to fasten the device on the tooth for the duration of the agent release (up to months). Using the holder attachment, even longer duration is preferred to facilitate longer treatments and easy changing of the device. The materials need to be biocompatible so that if the adhesive fails and the device is swallowed, no harm will result to the end-user.

At least one of the polymer matrices of the controlled release device according to the invention comprises an agent that can be released from the matrix in a controlled matter. The release of the agent is due to diffusion of the active agent, decomposition or dissolution of the polymer matrix of the device. The agent to be released from the polymer matrix can be a drug compound, a drug composition, a pharmaceutical component, a pharmaceutical adjuvant or additive, a mouth refresher agent or composition, a flavouring agent or composition, a sweetener agent or composition, colorants, nicotine, a vitamin or supplement, an active ingredient for weight loss, a peptide, a microparticle, a nanoparticle, or any combination thereof. In one embodiment the agent can be a drug, a pharmaceutical component, a mouth refresher agent or composition, a flavouring agent or composition, a sweetener agent or composition, a mucosal penetration or permeation enhancer, nicotine, vitamin, biomolecule, natural product, herbal extract, nanoparticle, microparticle, or other a combination thereof. The agent can be in solid form or liquid form or can be dissolved in a liquid. If the agent is in liquid form or dissolved in a liquid, the device is formed as a container or receptacle that can contain the liquid, or the liquid is absorbed into an additive, such as microcrystalline cellulose, and the additive is then loaded or incorporated into the device.

In one embodiment the agent is encapsulated or loaded into nano- or micro-particles and the nano- or micro-particles are embedded in the polymer matrix.

If the agent is embedded or encapsulated into the polymer matrix during the forming of the polymer matrix, the agent must withstand degradation during the polymer melting phase. Depending on the polymers, the temperatures during the melting phase vary from 50° C. to 450° C.

In one embodiment of the invention, the agent is a drug or pharmaceutical component. Preferred drugs or pharmaceutical components are drugs that are administered locally on or through the mucous membranes of the oral cavity and digestive tract, in order for the drugs or compounds to have a local or systemic effect. Other examples of preferred drugs include pharmaceutical components aimed at dementia patients or others that may have difficulties remembering to take the drug at the right intervals or pharmaceutical components that may require low dose sustained release to reduce side-effects.

In one embodiment of the invention, the agent is a drug or pharmaceutical component aimed at local treatment of diseases of the oral cavity and/or mucous membranes of the oral cavity and digestive tract. These local diseases include oral mucositis, oral or mouth cancer, graft-versus-host disease, oral bacteria infections, chronic inflammations of the mouth, mouth ulcers and aphthas. Suitable drugs usable as agents according to the invention include, but are not limited to, nonsteroidal anti-inflammatory drugs (NSAIDs), doxepin, L-glutamine, steroids (e.g., dexamethasone), antifungal and antibacterial drugs (e.g., clobetasol and ornidazole).

In one embodiment of the invention, the agent is nicotine. Nicotine treatment is commonly used for people aiming to quit or reduce smoking. Nicotine can be used alone or in combination with a mouth refresher agent or flavouring agent.

The controlled release device according to the invention can be in any suitable size. The thickness of the device can be from 0.1 mm to 2.5 mm, preferably from 0.5 mm to 2.0 mm and more preferably from 0.7 mm to 1.5 mm. The controlled release device can be formed as a capsule, a disc, a tablet, a cartridge, a pellet, a patch, a veneer or a troche depending on the end use of the device. If the device is formed as more or less a rectangular shape capsule or patch, the dimension of the sides of the rectangle can be from 3 mm to 15 mm, preferably from 3 mm to 10 mm, more preferably from 3 mm to 7 mm and most preferably from 3 mm to 5 mm. A typical device to be attached to a human tooth has the dimensions of 3.0 mm to 4.0 mm and a thickness of 1.5 mm. The dimensions of the controlled release device should be chosen such that the device is comfortable and preferably unnoticeable to the user.

The amount of agent in the controlled release device vary depending on the agent and the chosen release schedule of the agent. The amount of agent can vary from 0.5% to 40% of the total weight of the device. Preferably the amount of agent is from 1% to 20%, more preferably from 3% to 10% and even more preferably from 5% to 8% of the total weight of the device.

The controlled release device can be made by any method of preparing device from plastic materials, including casting, moulding and 3D-printing, for example. A film or a sheet of the polymer matrix used in the device can be formed and the controlled release devices will be cut out of the sheet or film. Alternatively, the devices can be printed using a 3D-printer suitable for printing objects of polymer materials. The holder can be also made by 3D-printing, injection molding or any other method suitable for the fabrication of 3D objects.

The controlled release device can be produced by incorporating the agent into the polymer matrix during the formation of the polymer matrix. The device is then manufactured by mixing the agent with the polymer material used in the polymer matrix. The polymer material is usually in the form of spherical or spheroidal pellets. The agent can also be processed with a spherodizer to prepare similar pellets and the pellets of the polymer can be mixed with pellets of the agent. Alternatively, the agent can be mixed as a powder with the polymer pellets when the agent powder adherers or is adsorbed on the surface of the polymer pellets. The mixture of agent and polymer pellets or the pellets on which the agent is adhered or adsorbed is then processed with a filament extruder that produces a polymer-agent filament of desired diameter.

The filament can be fed to a 3D-printer that melts the filament above the melting temperatures of the materials and prints the melt using a predetermined pattern to produce the agent containing the polymer layer or device. If the controlled release device comprises more than one polymer matrices each matrix can easily be printed as a separate layer of the device using a 3D-printer.

The controlled release device of the invention can also be produced by adding the agent to a polymer matrix already formed. Then the device is prepared by first making the device or part of the device by any polymer processing process and then soaking the device or polymer matrix in a solution of the agent. The agent will be absorbed by the polymer matrix and, thereby, embedded by the polymer matrix. Alternatively, the agent can also be adsorbed on the surface of the controlled release device or a polymer matrix layer.

### EXAMPLES

### Example 1

1.5 g of acetaminophen (paracetamol) was placed in a flask. 1 mL of water was evenly spread among the acetaminophen powder to moisten it. Then, a mixture of poly(lactide) (PLA) and poly(vinyl alcohol) (PVA) weighing 30 g in total was mixed with the moist drug powder. The amount of PVA was varied in the various mixtures accordingly; PVA 10 w-%, PVA 25 w-%, PVA 30 w-%, PVA 40 w-% and PVA 100 w-%, the balance being PLA. The mixture was stirred until all the drug has adhered on the surface of the polymer pellets. Then, the mixture was put into an oven at 40° C. to dry for 6 hours. Afterwards, the dried mixture was poured into the filament extruder operating at 170° C., which produced a filament with a thickness of 1.75±0.03 mm. The filament was fed to a 3D-printer operating at the nozzle temperature of 175° C. and nozzle diameter of 1.75 mm. The printer used a raster pattern to fabricate the devices layer-by-layer.

Each device or polymer-paracetamol formulation weighted 20 mg. With a paracetamol content of 5 w-% (1.5 g paracetamol on 30 g polymer), the maximum paracetamol release per device was estimated at 1 mg.

Three devices of each polymer matrix formulation were chosen for drug release experiments. Prior to the experiments, the dimensions and weight of the capsules were recorded. In the drug release experiments, the capsules were immersed in 2 mL of artificial saliva, which itself consists of a solution of 16.8 mM Na₂HPO₄, 1.4 mM KH₂PO₄, and 137 mM NaCl salts dissolved in purified water at pH 6.75. The solution was kept at 37° C. and under constant stirring. Once a day, 150 μL of solution was withdrawn from each sample to measure the amount of released drug in a high-performance liquid chromatography apparatus (HPLC). An equal amount of solution was added after each withdrawal to keep the total volume constant. The experiment was ceased between 35 and 42 days, due to devices turning red resulting from paracetamol degradation, or when all the drug was deemed to be released.

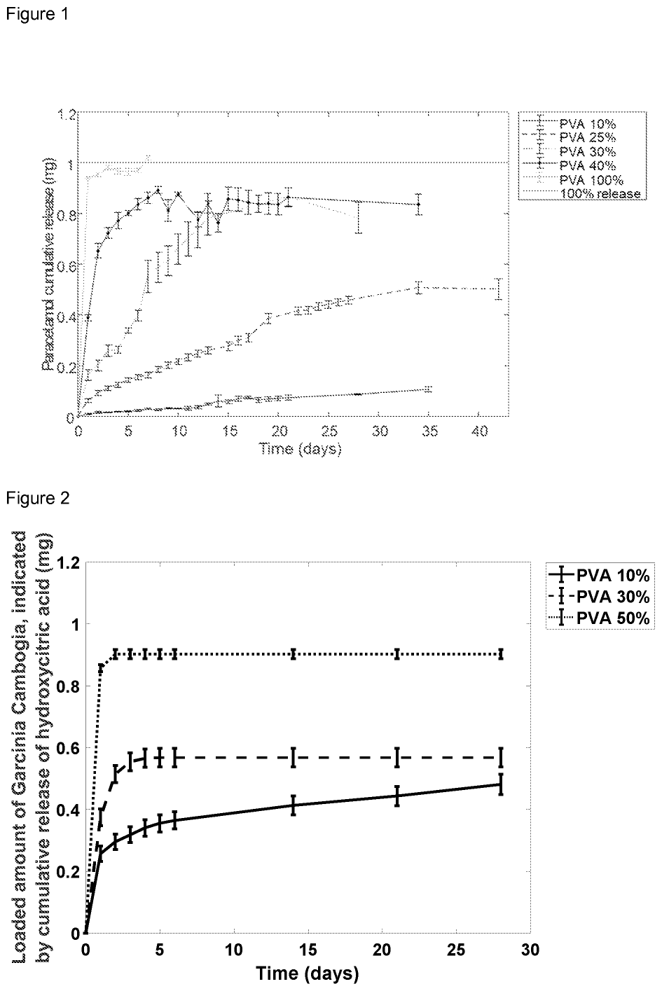

The results of the release test of paracetamol is shown in FIG. 1. In FIG. 1 it is shown that the release rate varies significantly depending on the amount of PVA. The release of the test agent paracetamol varies from almost complete release within a day when PVA content is 100 w-% of the polymer matrix to very limited release even within 25 days when PVA content is 10 w-% of the polymer matrix. It is therefore shown that the release rate can easily be controlled with varying the amount of PVA in the controlled release device.

### Example 2

3 g of Garcinia Cambogia herbal extract (Golden Horizon Biologics, the extract is based on an extraction of the fruit grind), comprising 60 w-% of hydroxycitric acid (HCA), was mixed with three different polymer matrices of PLA and PVA, each of the polymer matrices weighing in total 50 g, wherein the first one composed of 50 w-% of PVA, the second 30 w-% of PVA, and the third 10 w-% PVA, the balance being PLA. The 60 w-% content of HCA in the Garcinia Cambogia herbal extract was not verified. Thus the results are given in HCA released from an indicated amount of Garcinia Cambogia herbal extract loaded in a device. The subsequent fabrication procedure follows that of Example 1.

Each device weighed ca. 20 mg with estimated Garcinia Cambogia herbal extract content of 1.1 mg. Drug release experiments were performed to evaluate the loaded amount of Garcinia Cambogia herbal extract. This evaluation was done by measuring the released HCA, which directly gives the amount of Garcinia Cambogia herbal extract it originates from. The drug release experiments were performed similarly to those given in the Example 1, and results of the release experiments are shown in FIG. 2. The release was measured once per day for the first 6 days, after which samples were measured on days 14, 21, and 28.

The polymer matrix formulation with 50 w-% of PVA resulted in full HCA release in one day with a loading degree equivalent to HCA released from 0.9 mg of Garcinia Cambogia herbal extract loaded per device. The polymer matrix formulation with 30 w-% of PVA reached 100% release of HCA in three days, however, its loading degree was only about 0.6 mg Garcinia Cambogia herbal extract per device. The polymer matrix formulation with 10 w-% of PVA had a rapid release of HCA corresponding to a loaded amount of ca. 0.26 mg of Garcinia Cambogia herbal extract during the first day. Afterwards, the release rate was steady, reaching a released amount that corresponds to a loaded amount of ca. 0.48 mg Garcinia Cambogia herbal extract on day 28. These results further confirm that the release rate can be controlled by the relative amount of PVA and PLA. However, they also show that there may be burst release originating from the surface of the devices.

### Example 3

45 g of microcrystalline cellulose powder (50M, size) was placed into a flask. 40 mL of liquid nicotine was added into the flask to soak it into the cellulose. 15 g of vanillin was then mixed into the flask to conceal the odor of nicotine. Then, 20 g of PLA and 20 g of PVA were added into the flask to produce a polymer matrix formulation containing 50 w-% of PVA. Afterwards, the procedures are similar to Example 1.

Each device and formulation weighed ca. 20 mg, as in the previous examples. However, the loading degree of nicotine was 0.04 w-%, which is presumed to result from vaporization of nicotine in the filament extruder. The loading degree of vanillin was 0.32 w-%.

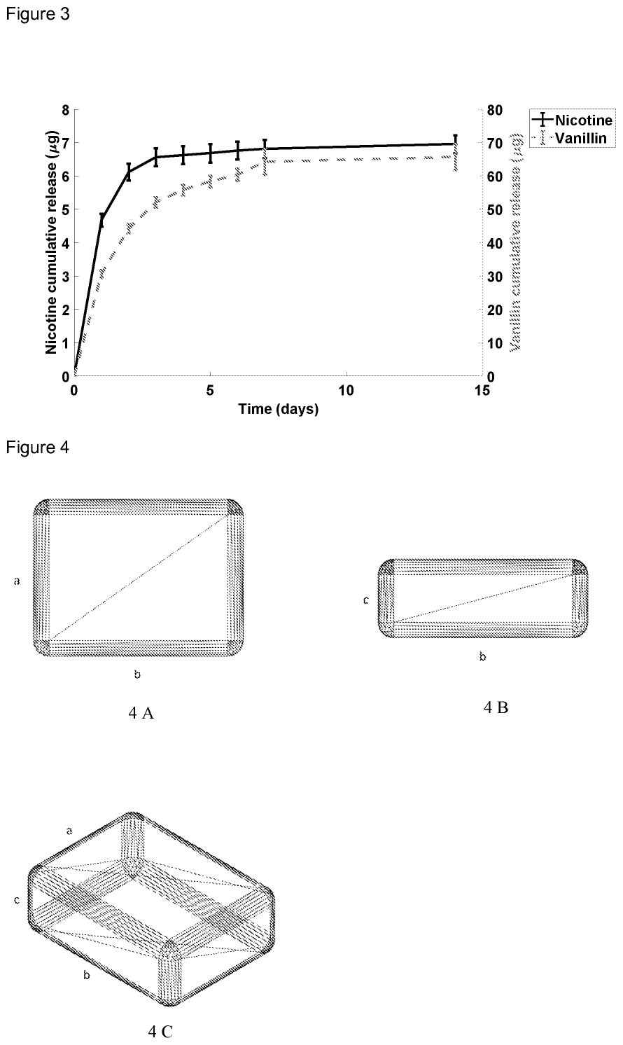

The release of nicotine and vanillin was measured similarly as in Examples 1 and 2. The results of the release experiments are shown in FIG. 3. Nicotine was released fast reaching 6 μg on day 2, after which the release slowed to result in ca. 7 μg on day 14. Vanillin reached ca. 50 μg release on day 4, after which it slowed to reach 65 μg on day 7. This result showed that the devices can release multiple ingredients simultaneously.

It will be obvious to a person skilled in the art that, as the technology advances, the inventive concept can be implemented in various ways. The invention and its embodiments are not limited to the examples described above, but may vary within the scope of the claims.

1. A controlled release device for delivering an agent to the oral cavity, wherein the device comprises
one or more polymer matrices,
each polymer matrix comprises one or more polymeric materials,
at least one polymer matrix comprises an agent that can be released from said matrix in a controlled manner,
means for attaching said device to a hard dental surface,
wherein at least one polymer matrix comprises at least two polymeric materials of which the first polymeric material is water insoluble and the second polymeric material is water soluble.

2. The device of claim 1, wherein said first polymeric material is poly(lactide) (PLA) or poly(caprolactone) (PCL).

3. The device according to claim 1, wherein said second polymeric material is poly(vinyl alcohol) (PVA) or poly(vinylpyrrolidone) (PVP).

4. The device according to claim 1, wherein the weight ratio of the first polymeric material to the second polymeric material is from 9:1 to 1:9, preferably from 8:2 to 2:8 and most preferably from 6:4 to 4:6.

5. The device according to claim 1, wherein said matrix is formed as a capsule, a disc, a tablet, a cartridge, a pellet, a veneer or a troche.

6. The device according to claim 1, wherein said device comprises at least two polymer matrices comprising an agent that can be released from said polymer matrix in a controlled manner and the polymer matrices each comprise the same agent or different agents.

7. The device according to claim 1, wherein at least one polymer matrix comprises one or more further components.

8. The device according to claim 1, wherein said agent is selected from a drug, a pharmaceutical component, a mouth refresher agent or composition, a flavouring agent or composition, a sweetener agent or composition, a mucosal penetration or permeation enhancer, nicotine, vitamin, biomolecule, natural product, herbal extract, nanoparticle, microparticle or a combination thereof.

9. The device according to claim 1, wherein said agent is a liquid or the agent is dissolved in a liquid and the liquid is encapsulated or loaded in said device.

10. The device according to claim 1, where the agent is encapsulated or loaded into nano- or micro-particles and the nano- or micro-particles are embedded in the polymer matrix.

11. The device according to claim 1, wherein said means for attaching said controlled release device to a hard dental surface is selected from
at least one of the polymeric matrices forms an adhesive layer that attaches said controlled release device to the hard dental surface,
a holder attached to the hard dental surface and to which holder the device can be attached, clipped or fastened, and
an adhesive material that forms a layer on the controlled release device and can attach the device to the hard dental surface.

12. A method of producing a device according to claim 1 comprising the steps of
forming a polymer matrix from at least two polymeric material,
adding to said polymer matrix an agent,
optionally adding further components to the polymer matrix and
optionally forming the polymer matrix including the agent to a capsule, a disc, a tablet, a cartridge, a pellet, a veneer or a troche.

13. A method of producing a device according to claim 1 comprising the steps of
incorporating an agent into a polymeric material during formation of a polymer matrix from at least two polymeric material,
optionally adding further components to the polymer matrix and
optionally forming the polymer matrix including the agent to a capsule, a disc, a tablet, a cartridge, a pellet, a veneer or a troche.

14. The device according to claim 1, wherein the weight ratio of the first polymeric material to the second polymer material depends on the desired release rate of the agent.
